(19) United States
(12) Patent Application Publication
(10) Pub. No.: US 2015/0374255 A1
(43) Pub. Date: Dec. 31, 2015

(54) Adhesive-Mountable Head-Wearable EEG Apparatus

(71) Applicant: Curzio Vasapollo, Tokyo (JP)

(72) Inventor: Curzio Vasapollo, Tokyo (JP)

(21) Appl. No.: 14/318,668

(22) Filed: Jun. 29, 2014

(51) Int. Cl. A61B 5/0478; A61B 5/00; A61B 5/1455

(52) U.S. Cl. CPC A61B 5/0478; A61B 5/14551; A61B 5/6814

(57) ABSTRACT

An adhesive-mountable head-wearable EEG apparatus is disclosed. The apparatus includes an EEG sensor for acquiring an EEG signal of a wearer, a central processing unit for receiving the EEG signal, a small circuit board including the EEG sensor and the central processing unit, and a compact enclosing shell for enclosing the small circuit board, the EEG sensor, and the central processing unit. An adhesive electrode assembly attaches to the compact enclosing shell, or to the small circuit board within the enclosing shell, via snaps or magnets. The adhesive electrode assembly includes two or more gel electrodes for acquiring an EEG signal, and for adhering to the forehead so as to wearably support the EEG apparatus on the forehead. The compact enclosing shell includes chamfered edges, and is sized so as to reduce lateral forces on the compact shell that would tend to detach the EEG apparatus from the wearer's forehead.

### FIELD OF THE INVENTION

This invention relates to head-wearable physiological monitoring devices, and particularly to such devices that include EEG monitoring.

### BACKGROUND OF THE INVENTION

Physiological monitoring of a kind and accuracy once possible only in a clinical setting and with the aid of trained medical staff is becoming available as wearable consumer devices. This phenomenon promises to bring many benefits. First, lowered costs for patients. Second, the possibility to acquire physiological measurements over a longer time span, with benefits for both research and diagnosis. Third, the possibility of monitoring physiological parameters in a “real life” setting as opposed to an artificial laboratory setting. Fourth, the possibility to monitor physiological parameters with less discomfort, without disturbing the patient and compromising the data being acquired. Fifth, because of lowered costs and increased comfort, the benefits of physiological monitoring and early diagnosis can be extended to users with mild or no symptoms, and who would not under ordinary circumstances have sought physiological monitoring in a medical setting.

However, all the benefits listed above are dependent on adoption and use of such consumer devices by the end user; and in turn these depend heavily on form factor, absence of wires, ease of operation, and comfort.

EEG monitoring normally involves affixing multiple wired electrodes to a patient's head in a clinical setting. When physiological parameters other than EEG are also acquired, more sensors must be affixed to the patient's body. For instance, in the case of pulse oximetry, a finger clip is used. Wired sensors are disliked by patients, and when they are used during sleep they disturb the process they are meant to monitor. Wired sensors are usually also connected to bulky machinery. Further, wired sensors are delicate, and can become dislodged easily when the patient moves; their use normally requires trained staff.

It is perhaps due to these difficulties that sleep test devices designed specifically for home use, such as the Clevemed® SleepView™, Novasom® Accusom™, the Watermark® ARES™ and others, do not include EEG sensors.

The Zeo™ headband by Zeo, Inc. (now out of production) was one of the first compact head-wearable EEG monitoring devices, directly connected to a textile-based conductive headband, and worn on a subject's forehead during sleep. It did not seek to measure any physiological parameter other than EEG. It used snap buttons embedded in the enclosure to connect the device to a replaceable textile-based electrode headband.

Due to the high impedance of the conductive textile sensors used in the Zeo™, users reported inaccurate readings, high noise, and poor electrode performance. Furthermore, users with long hair reported problems with the headband's stability and comfort.

In the consumer space, after the demise of the Zeo™, many more consumer-grade wearable EEG devices have become available, such as Neurosky™ “headset” type EEGs, Emotiv® Epoc™, the Melon™ headband, the InteraXon® Muse™ and others. None are suitable for wearing while the subject is sleeping due to their construction. The Neurosky™ device looks like a headset and requires an ear clip electrode. The Epoc™ device has delicate electrodes all over the wearer's head. The Melon™ headband has only one channel, and the thickness of the device would make it difficult to wear during sleep. The Muse™ hides the bulk of the device behind the ears, again making it unsuitable for wearing during sleep.

### SUMMARY

The EEG apparatus of the invention improves the state of the art by adding comfort and user-friendliness, and providing a higher degree of miniaturization.

Because the EEG apparatus of the invention has an adhesive electrode assembly including multiple electrodes, applying the entire assembly results in the application of multiple electrodes, improving ease of use, user-friendliness, and reducing the likelihood of one of the electrodes becoming disconnected. Furthermore, the adhesive electrode assembly allows simultaneous acquisition of multiple EEG channels. For sleep-staging purposes, even if one part of the electrode assembly becomes disconnected, the remaining channel(s) can be used to stage sleep.

Due to the use of snap buttons or magnetic attachments, no wires are needed.

Due to the presence of a hole or a slit in the electrode assembly, pulse oximetry can also optionally be measured from the forehead through the hole or slit, without the need to use a traditional wired finger sensor.

Unlike conductive fabrics, regular gel electrodes offer good impedance characteristics and yield low noise signals. Further, the electrode assembly of the invention exploits the adhesiveness of gel electrodes. According to the invention, a plurality of electrodes can support and mechanically mount a compact EEG device to the forehead of a wearer in the case of a compact and light-weight device having a small circuit board and an enclosing shell weighing only a few grams, thereby making a supporting headband unnecessary in most cases.

The EEG apparatus of the invention is multi-channel, low noise, wearable during sleep, has a favorable compact and light-weight form factor for adoption by consumers, can be applied to the wearer's forehead in one simple operation without assistance, and allows easy acquisition of pulse oximetry in addition to EEG.

A general aspect of the invention is an adhesive-mountable head-wearable EEG apparatus for EEG monitoring. The apparatus includes: an EEG sensor capable of acquiring an EEG signal of a wearer of the wearable EEG apparatus; a central processing unit capable of receiving the EEG signal from the EEG sensor; a circuit board including the EEG sensor and the central processing unit; an enclosing shell for enclosing the circuit board, the EEG sensor, and the central processing unit; and an adhesive electrode assembly. The adhesive electrode assembly includes: two or more gel electrodes capable of acquiring an EEG signal and capable of adhering to the forehead, two or more respective electrically conductive connection elements, each connection element being electrically connected to one of the gel electrodes, each connection element additionally being capable of being mated with a corresponding connection element on one of: the enclosing shell or the circuit board, so as to both electrically connect the circuit board to the electrodes, and structurally support the enclosing shell.

In some embodiments, the adhesive electrode assembly further comprises at least one adhesive non-conductive area.

In some embodiments, the enclosing shell has a left chamfered edge and a right chamfered edge, so as to reduce lateral forces on the enclosing shell when the apparatus is worn during sleep.

In some embodiments, a horizontal width of the enclosing shell is no greater than a width that would bring the edge of the enclosing shell in contact with a sleep surface supporting the wearer before the nose of the wearer contacts the sleep surface.

In some embodiments, the depth of the enclosing shell is no more than 2 cm.

In some embodiments, the connection elements are snap fasteners.

In some embodiments, the connection elements are magnets.

In some embodiments, the adhesive electrode assembly further includes: an aperture capable of allowing light to be directed towards and reflected by the forehead of the wearer, so as to allow a reflectance oximetry sensor to acquire oximetry measurements from the forehead while the electrode assembly is affixed to the forehead.

In some embodiments, the gel electrodes are Ag—AgCl gel electrodes.

Another general aspect of the invention is a method of simultaneously affixing and electrically connecting a forehead-worn EEG device to a person's forehead. The method includes: connecting an adhesive electrode assembly to the forehead-worn EEG device using two or more contact elements; and affixing the adhesive electrode assembly to the forehead, thereby creating two or more respective electrical connections between the person's forehead and the forehead-worn EEG device.

In some embodiments of the method, the contact elements are snap fasteners.

In some embodiments of the method, the contact elements are magnets.

In some embodiments of the method, the method further includes: capturing a headband between the forehead-worn EEG device and the electrodes, so as to enable the headband to support the electrode assembly and ensure its adherence to the forehead.

Another general aspect of the invention is a method of simultaneously acquiring EEG and pulse oximetry from the forehead of a person. This method includes: connecting a multi-polar adhesive electrode assembly to a multi-sensor forehead-worn device using two or more contact elements; affixing the electrode assembly to the forehead, thereby creating two or more distinct electrical connections between the forehead and the device; acquiring an EEG signal by amplifying voltages measured on the electrical connections; emitting light from the sensing device, such that the light reaches the forehead through an aperture in the electrode assembly; and measuring the intensity of light reflected by the forehead.

### BRIEF DESCRIPTION OF THE DRAWINGS

The invention will be more fully understood by reference to the Detailed Description, in conjunction with the following figures, wherein:

FIG. 1 is a line drawing of the front of an adhesive electrode assembly, the adhesive electrode assembly including four male snap buttons and a hole.

FIG. 2 is a line drawing of the back of the adhesive electrode assembly.

FIG. 3 is a line drawing showing another frontal view of the adhesive electrode assembly.

FIG. 4 is a line drawing of an alternate embodiment of the adhesive electrode assembly of FIG. 1, this embodiment having no hole in the center.

FIG. 5 is a line drawing of an alternate embodiment of the adhesive electrode assembly of FIG. 1, this embodiment having only three male snap buttons.

FIG. 6 is a line drawing of an EEG monitoring device.

FIG. 7 is a line drawing of an alternate embodiment of the adhesive electrode assembly of FIG. 1, this embodiment having female snap buttons.

FIG. 8 is a line drawing of an alternate embodiment of the adhesive electrode assembly of FIG. 1, this embodiment including a slit instead of a hole.

FIG. 9 is a line drawing of an alternate embodiment of the adhesive electrode assembly of FIG. 1, this embodiment including magnets instead of snap buttons.

FIG. 10 is a line drawing of the adhesive electrode assembly of FIG. 7, connected to the EEG monitoring device of FIG. 6 and affixed to a patient's forehead

FIG. 11 is a line drawing of the adhesive electrode assembly and EEG monitoring device of FIG. 10, also including a headband that fully occludes the electrode assembly.

FIG. 12 is a line drawing of the first step of the mounting of the headband of FIG. 11; in this step, two holes on the headband are positioned above two of the snap buttons on the EEG monitoring device.

FIG. 13 is a line drawing of the second step of the mounting of the headband; in this step, the adhesive electrode assembly is connected to the EEG monitoring device by means of snap buttons on the adhesive electrode assembly and snap buttons on the device.

FIG. 14 is a schematic split view of the enclosure of the EEG monitoring device, the adhesive electrode assembly and the wearer's forehead epidermis. FIG. 14 schematically illustrates the acquisition of the pulse oximetry signal.

FIG. 15 is a schematic split view of the EEG monitoring device and the adhesive electrode assembly, when the snap buttons have been replaced by magnetic elements such as neodymium magnets.

FIG. 16 is a schematic view of another embodiment of the adhesive electrode assembly, this embodiment including two non-conductive adhesive areas.

FIG. 17 is a schematic view of another embodiment of the adhesive electrode assembly, this embodiment including Ag—AgCl electrodes and a large non-conductive adhesive area.

FIG. 18 is a schematic diagram of the components of an embodiment of a head-wearable EEG apparatus of the invention.

FIG. 19 is a line drawing of a view from above of the EEG monitoring device of FIG. 6 when the device is worn on the forehead, showing the right and left chamfered edges of the device.

FIG. 20 is a line drawing of a sleeping person wearing the EEG monitoring device of the invention.

FIG. 21 is a flow diagram of the steps of a method of simultaneously affixing and electrically connecting a forehead-worn EEG device to a person's forehead.

FIG. 22 is a flow diagram of the steps of a method of simultaneously acquiring EEG and pulse oximetry from the forehead of a person.

### DETAILED DESCRIPTION OF THE INVENTION

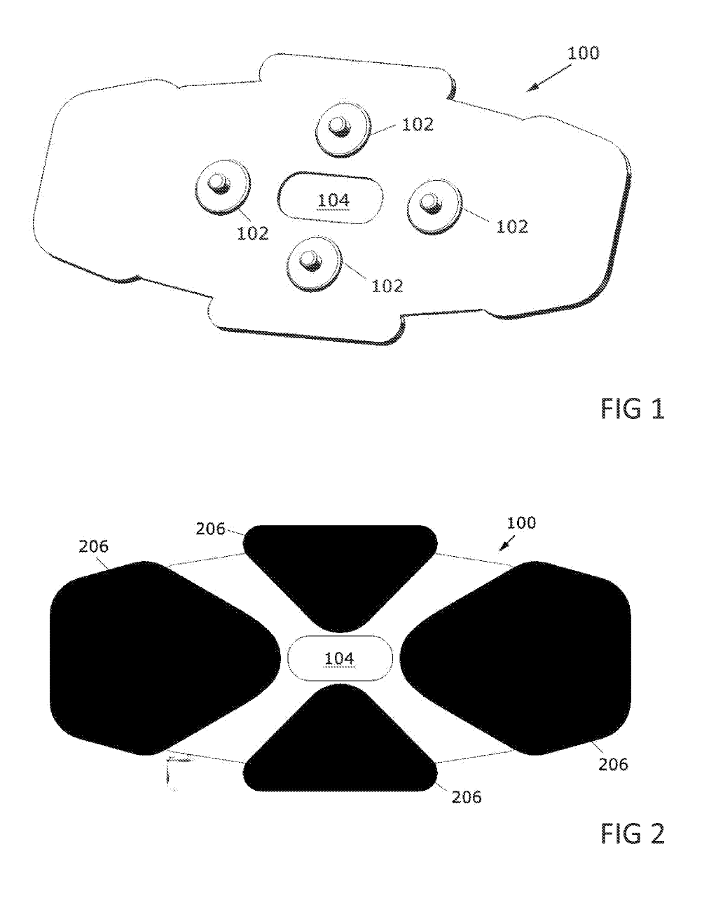

With reference to FIGS. 1-3, an adhesive electrode assembly 100 has four connection elements 102. A single adhesive electrode assembly 100 can be affixed to the forehead of a wearer easily and reliably even by inexperienced users, and reduces the possibility of individual electrodes becoming disconnected. The EEG signal is acquired from the wearer's forehead through the four gel electrodes 206. The electrical potential on the left and right gel electrodes 206 is measured against a reference electrode (either the central top or central bottom electrode 206). The remaining central top or bottom electrode 206 (the electrode not used as reference electrode) is an output from the EEG monitoring device, having “right leg drive” function to reduce common mode noise. Therefore the four electrodes 206 are used to acquire the left and right hemisphere frontal EEG signal (two channels of data), with low noise.

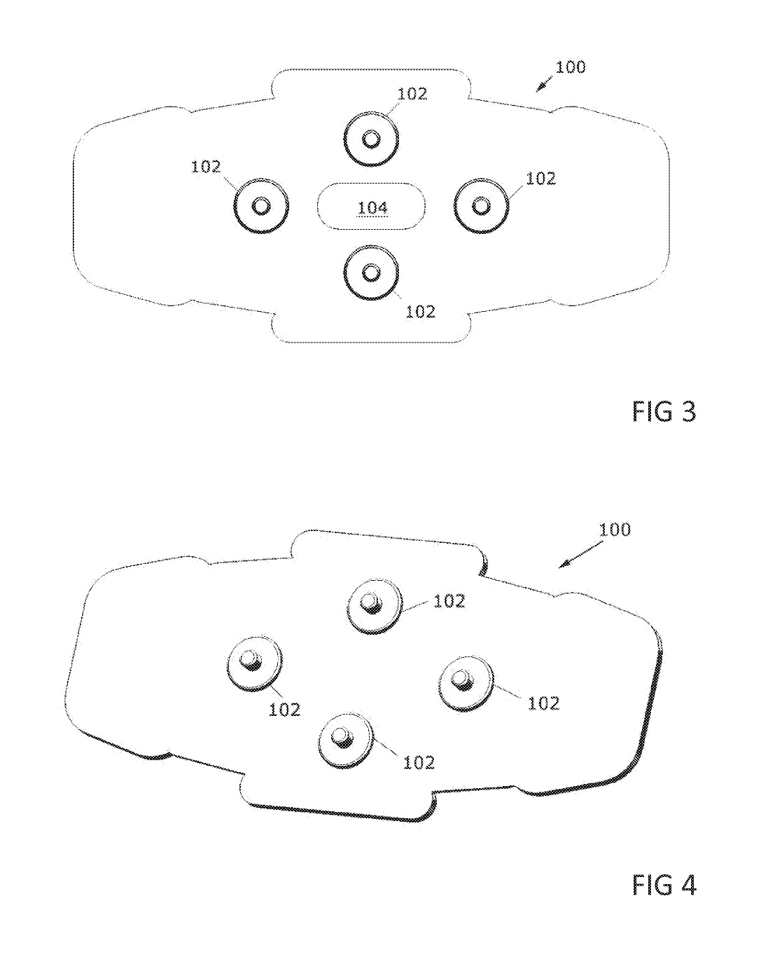

FIG. 4 shows an alternate embodiment of the adhesive electrode assembly of FIG. 3, this embodiment having no hole in the center.

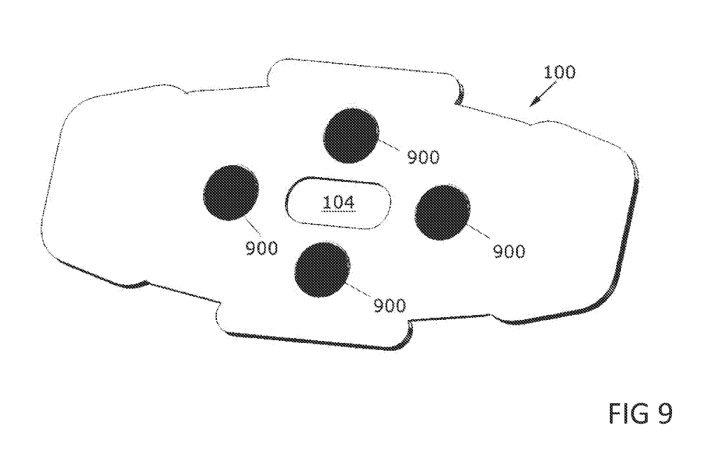

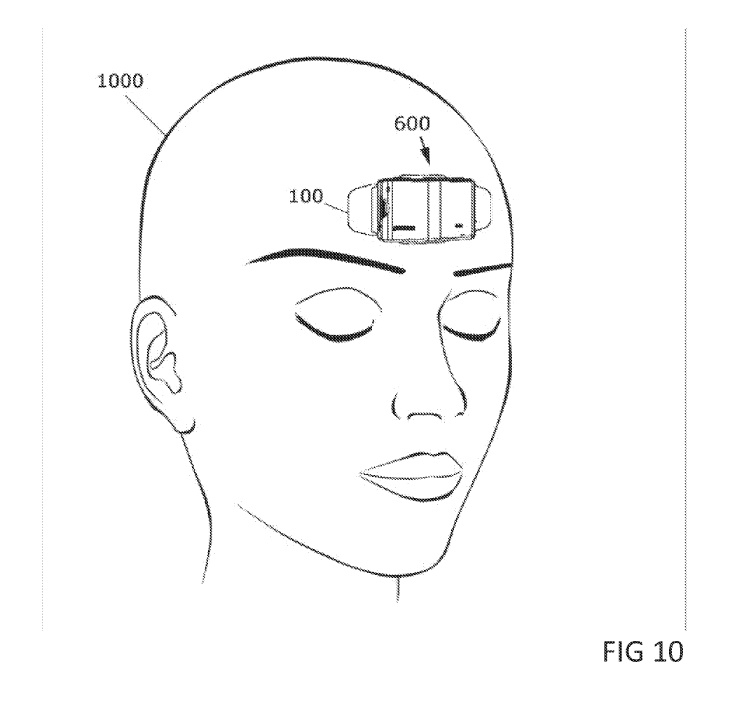

It is possible to reduce the number of electrodes by one, by eliminating the right leg drive, and in this case either one snap button 102 or one of the gel electrodes 206 or both can be optionally dispensed with. However, both the snap button and the electrode can also be included (though unused) so as to provide increased strength of adhesion to the forehead, and mechanical strength of the structural connection between the electrode assembly and the EEG monitoring device. Retaining mechanical strength would be an important consideration in case the snap buttons are replaced with magnets as in the embodiment of FIG. 9.

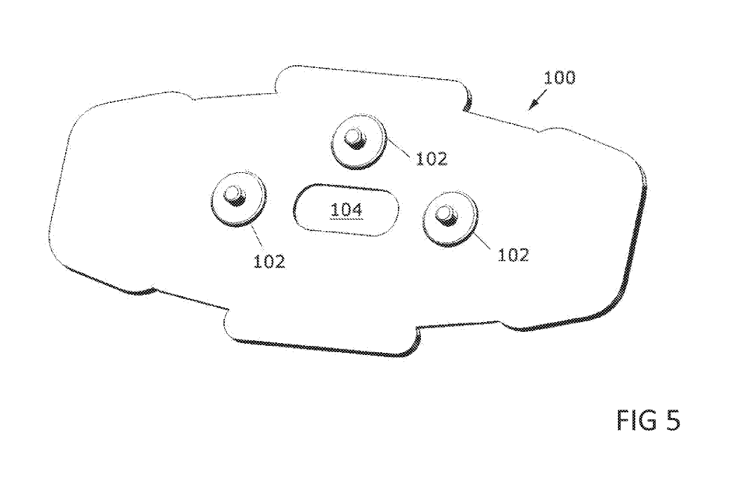

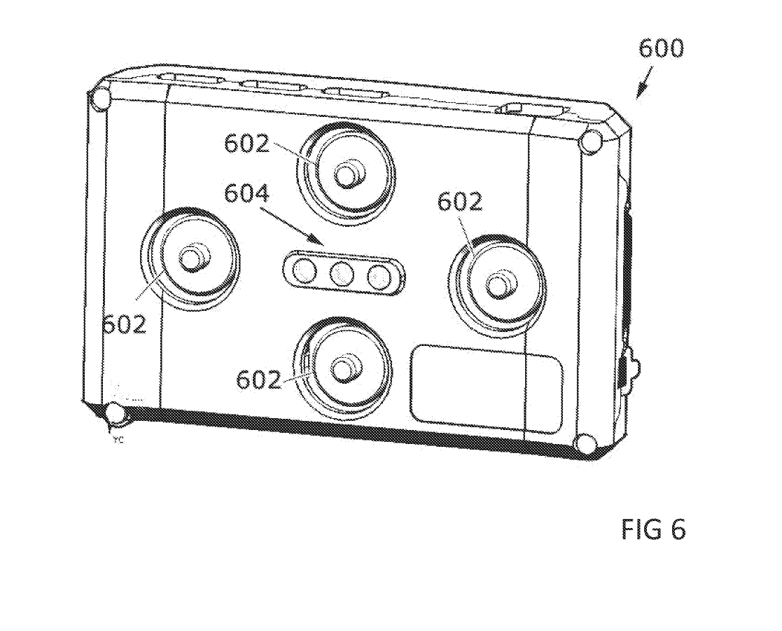

FIG. 5 shows an adhesive electrode assembly 100 with only three snap buttons 102, as may be utilized when right leg drive is not needed. Dispensing with the right leg drive, however, would adversely impact the noise characteristics of the device.

It is also possible to eliminate one of the EEG channels, thereby only acquiring one channel of EEG data, and this would also reduce the electrode count by one, making the lowest possible number of contact points 2. However this would reduce the ability of the EEG monitoring device to detect proper electrode contact. Both changes in skin hydration (such as perspiration or drying) and electrode placement can affect the impedance of each pair of electrodes used for monitoring a channel of EEG. With two channels, changes in skin hydration and changes in electrode impedance due to improper placement or the electrodes peeling off can be discriminated. Changes in skin hydration will yield an identical change in impedance on both channels, whereas a change in the adhesion area of one of the electrodes for a given channel will only be reflected on the impedance of that respective channel.

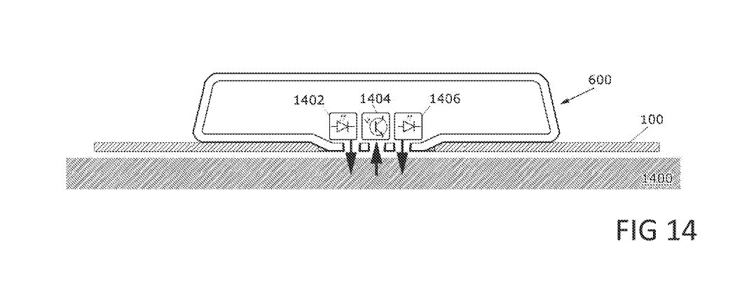

In FIG. 2 a hole 104 in the adhesive electrode assembly 100 allows light emitted by the device (not shown) to reach the forehead, and the light reflected from the forehead to be measured by a sensor within the device. This assembly therefore constitutes a reflectance oximeter. FIG. 14 illustrates the components of the oximeter when an EEG monitoring device 600 is attached to the electrode assembly 100, and the electrode assembly 100 is affixed to the wearer's forehead 1400. According to commonly used reflectance pulse oximetry, a red LED 1402 and an infrared LED 1406 are used to send light into the forehead 1400 of the wearer. Light is reflected back to a sensor, such as a phototransistor 1404, and measured.

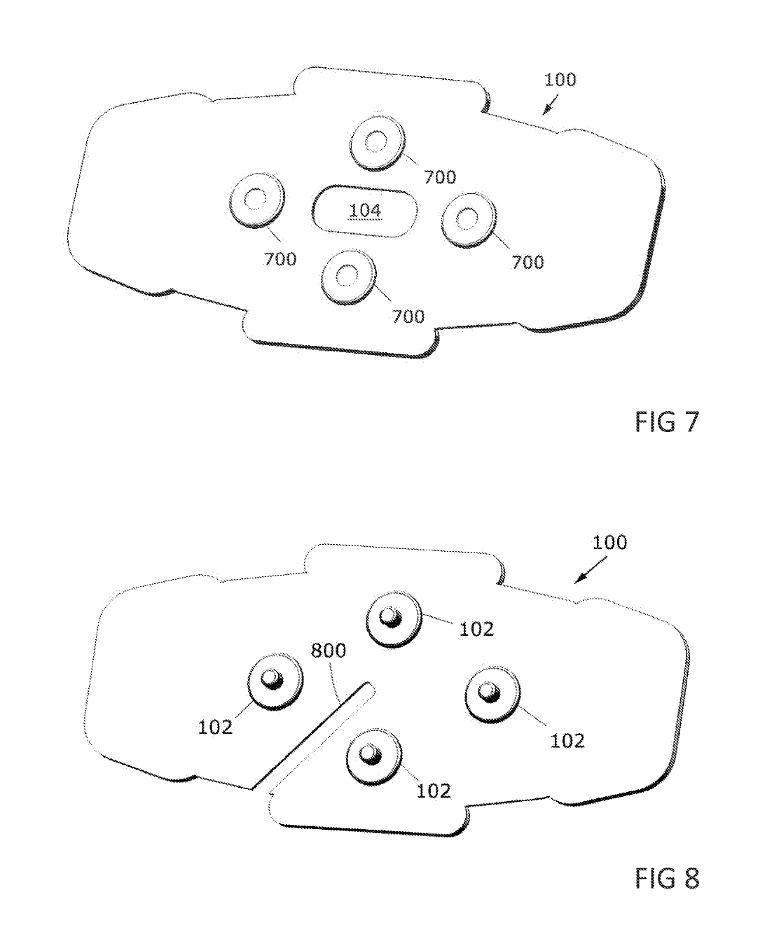

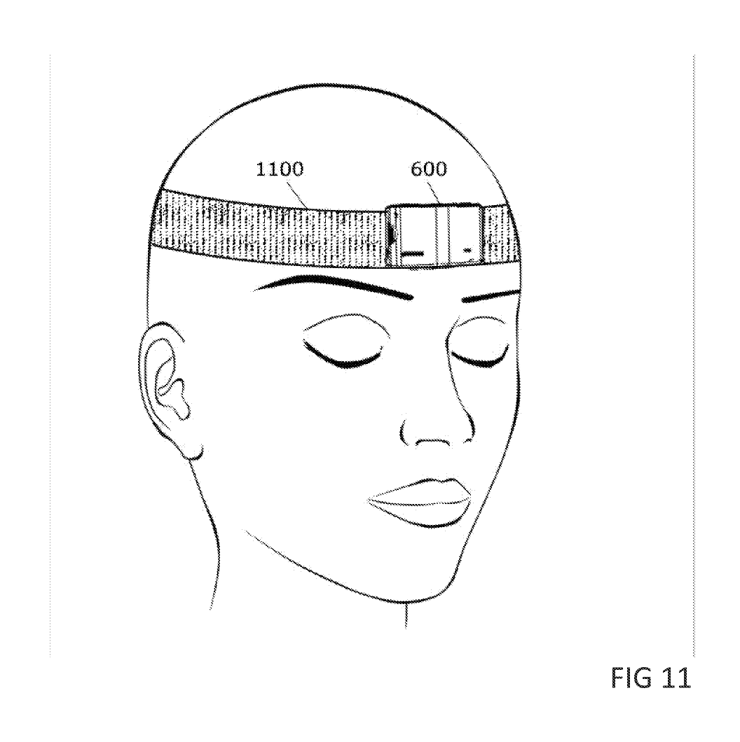

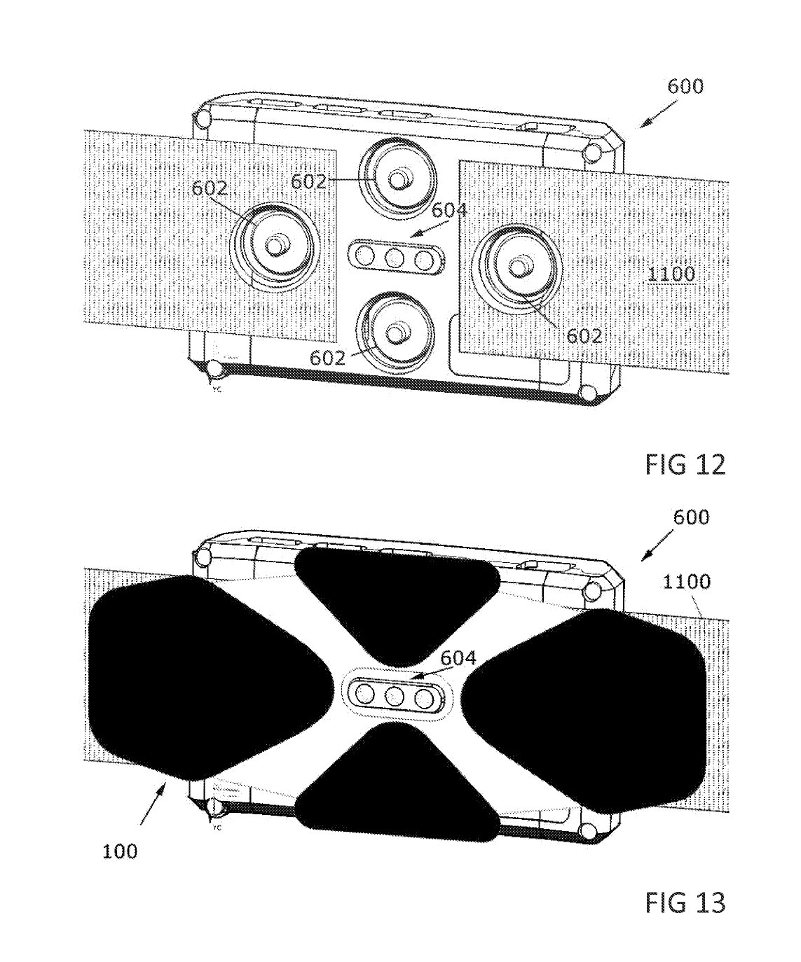

The adhesive electrode assembly 100 has sufficient adhesive surface area to allow the device to adhere to the forehead during EEG monitoring. It is possible however to use a headband 1100 (FIG. 11) to provide further reinforcement to hold the device 600 in place. With reference to FIG. 12, a suitable headband has a hole on each end. The holes are positioned above the left and right connection elements (snap buttons) 602 on the device. When the snap buttons on the electrode assembly 700 (FIG. 7) are mated with those 602 on the device (FIG. 12), the headband is therefore held in place.

In FIG. 8, a slit 800 in the adhesive electrode assembly 100 is used to acquire oximetry measurements. A matching EEG monitoring device 600 to this embodiment of the adhesive electrode assembly 100 would have an oximetry sensor 604 positioned so that light to and from the oximetry sensor 604 can reach the forehead of the wearer passing through the slit 800.

Furthermore, even without a hole 104 or a slit 800, pulse oximetry can be acquired from the forehead by increasing the size of the EEG monitoring device 600 so that it extends beyond the electrode assembly, or dispensing with one of the electrodes (thus reducing the size of the electrode assembly) or by other means, so long as the pulse oximetry LEDs and sensor can have access to the skin of the forehead. However, the preferred embodiment has the pulse oximeter positioned above a hole 104 in the electrode assembly, so as to maintain the distance between the skin and the oximetry sensor constant, avoid ambient light interference, keep the device size minimal, and accommodate four electrodes.

The difference between FIG. 7 and FIG. 1 is the gender of the snap buttons used as connection elements. Snap buttons on the electrode assembly 700 must be of opposite gender to snap buttons 602 on the EEG monitoring device 600.

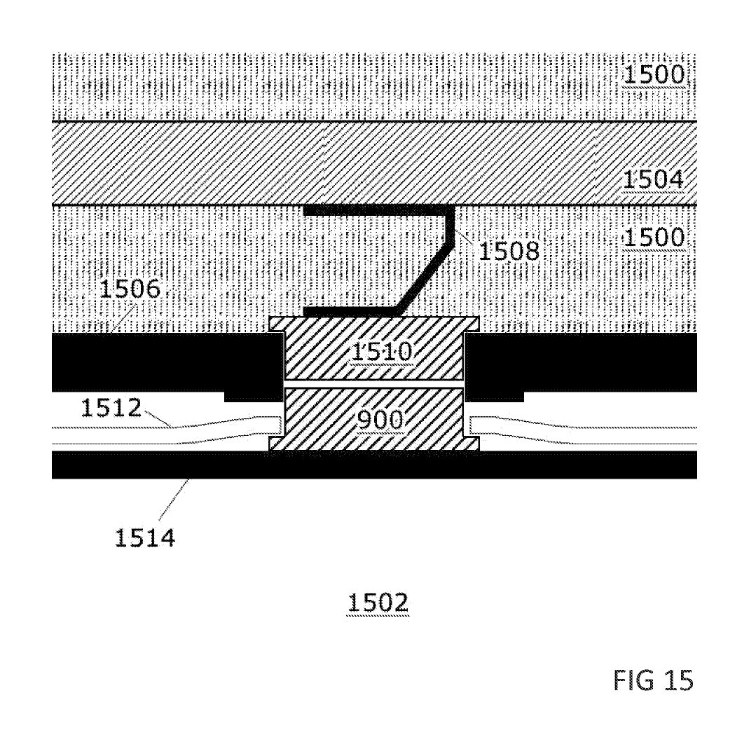

With reference to FIG. 9, magnetic elements 900 (for instance, simple neodymium magnets in contact with the gel electrodes below) can be used instead of snap buttons, providing further ease of use, and yet sufficient mechanical strength when the adhesive electrode assembly 100 is attached to the EEG monitoring device 600. Unlike snap buttons, neodymium magnets are not suited to being soldered directly onto a circuit board, because they lose most of their magnetic properties when heated to the necessary temperatures. To use magnetic contact points, magnets must be mounted on the circuit board by means other than soldering. Though using conductive glue or tape is an option for prototype production, it is not a good production strategy for large quantities of devices. Spring contacts can instead be soldered directly onto the circuit board during automated assembly, and the magnets inserted into properly sized holes in the plastic shell. A viable system of utilizing magnetic contacts to attach the electrode assembly to the EEG monitoring device is shown in FIG. 15. In this figure, the textured/dotted area represents the inside 1500 of the enclosing shell, and the white area the outside space 1502. The adhesive electrode assembly 100 includes two layers; a textile layer 1512 (normally a non-woven white textile material common to most ECG/TENS electrodes) and a conductive gel layer 1514. A neodymium magnet or other magnetic element 900 is embedded in the adhesive electrode assembly so that it is in contact with the conductive gel layer 1514. This electrode-side magnetic element 900 has a disk-shaped base so that it can be held in place by the overlying textile 1512. The electrode-side magnetic element 900 mates with a device-side magnetic element 1510. The device-side magnetic element 1510 is embedded in the plastic enclosure 1506 of the EEG monitoring device. The plastic shell 1506 has a hole of matching shape and size, into which the magnetic element 1506 is inserted. Once inserted, the magnetic element 1506 is unable to escape from the shell towards the outside, due to its shape. Contact with the circuit board 1504 is achieved by means of a spring contact 1508. The advantage of this embodiment is decreased stress to the components mounted on the circuit board when electrode assembly is attached to the device or removed. From the perspective of a user, it is also a somewhat more stylish and desirable embodiment. However, the snap button mechanism is a reliable one with little complexity, and electrode manufacturers are already familiar with embedding snap buttons into gel electrodes; furthermore no custom shaped magnets need to be created, so the snap button solution is in many instances more practical and has lower cost. It is to be noted that a plastic enclosing shell can be able to accommodate a cylindrical magnet, holding it in place by interference alone once inserted, whereas the electrode assembly is composed of soft material and therefore at least on the electrode side, a magnetic element having a disk-shaped base as shown in FIG. 15 is preferred.

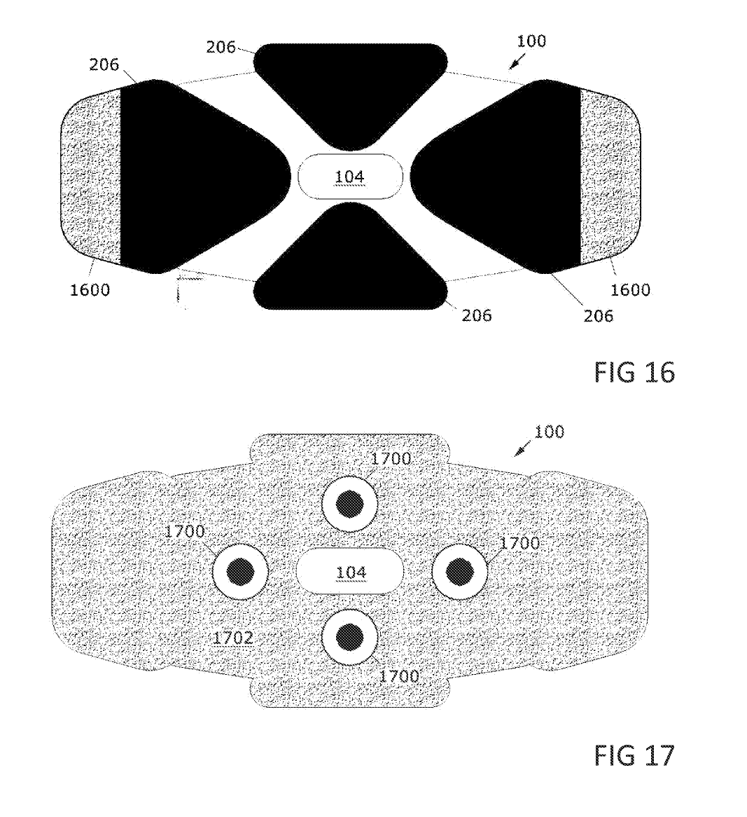

FIG. 16 shows an alternate embodiment of the electrode assembly 100 of FIG. 1 and FIG. 2, this embodiment including two non-conductive adhesive areas 1600. Although the gel electrodes 206 are themselves adhesive, the adhesiveness of the gel is not as strong as that of common adhesive.

The two non-conductive adhesive areas 1600 increase the adhesiveness of the electrode assembly 100, thereby preventing the electrode assembly 100 from peeling off of the patient's 1000 forehead even when the patient is wearing the EEG apparatus during sleep, and tossing/turning in bed.

The two non-conductive adhesive areas 1600 are portions of the electrode assembly 100 in which the top white layer is coated with an adhesive, a feature common to many ordinary snap gel electrodes.

FIG. 17 shows an alternate embodiment of the electrode assembly 100 of FIG. 1 and FIG. 2, including four Ag—AgCl gel contact points 1700 instead of the regular gel contact points 206. Because Ag—AgCl gel contact points are quite small, in the embodiment of FIG. 17 adhesion is provided by a single, large non-conductive adhesive area 1702 occupying most of the surface area of the electrode assembly.

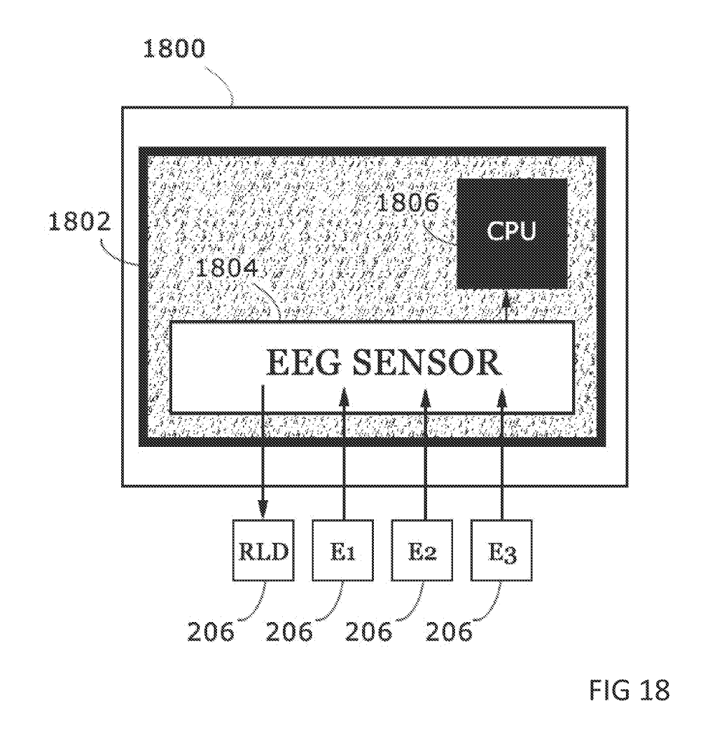

FIG. 18 shows the constituent components of the EEG apparatus of claim 1. An enclosing shell 1800 encloses a circuit board 1802, a CPU 1806, and an EEG sensor 1804. The EEG sensor 1804 is connected to four electrodes 206; one right leg drive output electrode and three input electrodes for acquiring two EEG channels. The CPU 1806 acquires an EEG signal from the EEG sensor 1804.

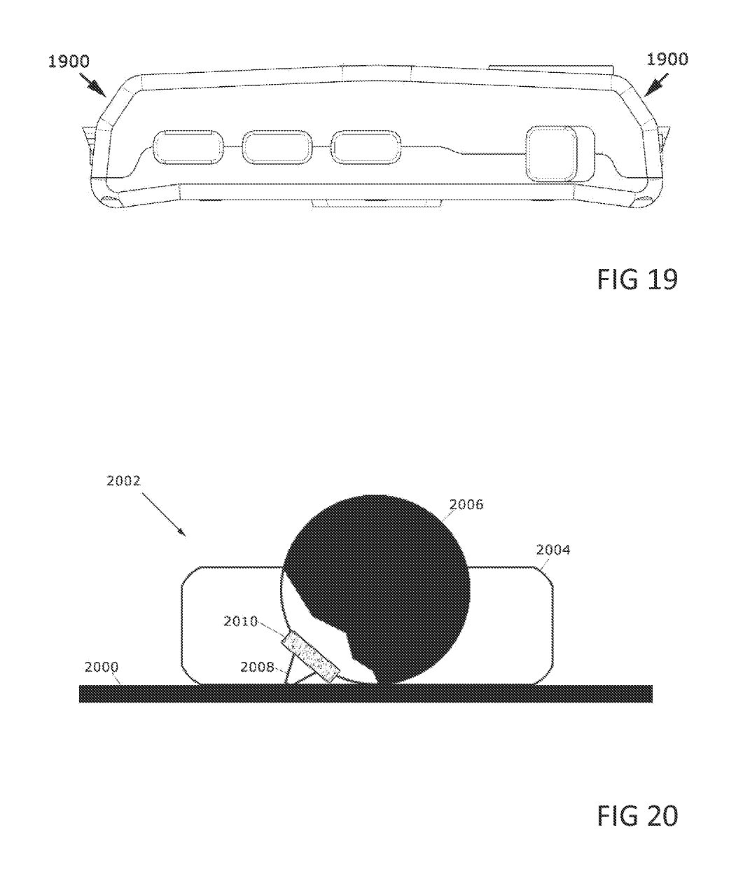

FIG. 19 shows the top view of the enclosing shell of the EEG monitoring device, as seen looking down on the top of a wearer's head. The enclosing shell has right and left chamfered edges 1900 so as to reduce lateral forces on the enclosing shell when the apparatus is worn during sleep.

FIG. 20 shows a sleeping person 2002. The person 2002 is sleeping prone, his body 2004 supported by a sleep surface 2000. The person's head 2006 is directly resting on the sleeping surface 2000, oriented downwards to the extent that the person's nose 2008 will permit. The EEG monitoring apparatus can be worn without interfering with the person's 2002 sleeping position as long as the horizontal width between the two chamfered edges 1900 of the shell of the EEG monitoring device 2010 is such that the chamfered edge of the EEG monitoring device 2010 does not come in contact with the sleeping surface 2000 before the nose as the person 2002 rotates his/her head 2006 during sleep, in the process of assuming the sleeping position shown in FIG. 20.

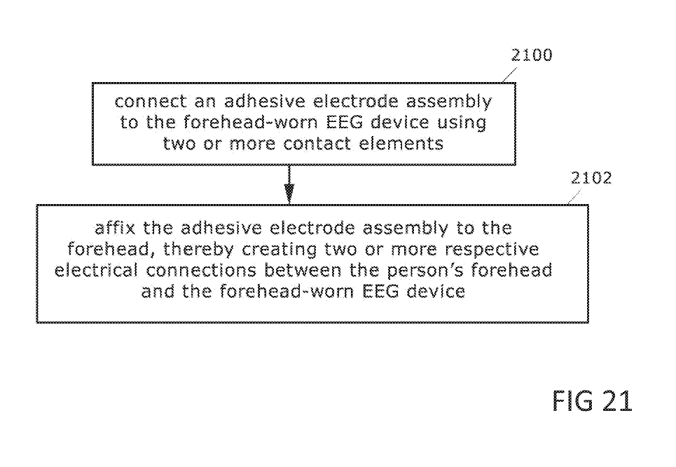

FIG. 21 shows the steps of a method for simultaneously affixing and electrically connecting a forehead-worn EEG device to a person's forehead. In the electrode connection stage 2100, the adhesive electrode assembly 100 is connected to the EEG monitoring device 600. In the wearing stage 2102, the adhesive electrode assembly 100 is affixed to the forehead of a wearer.

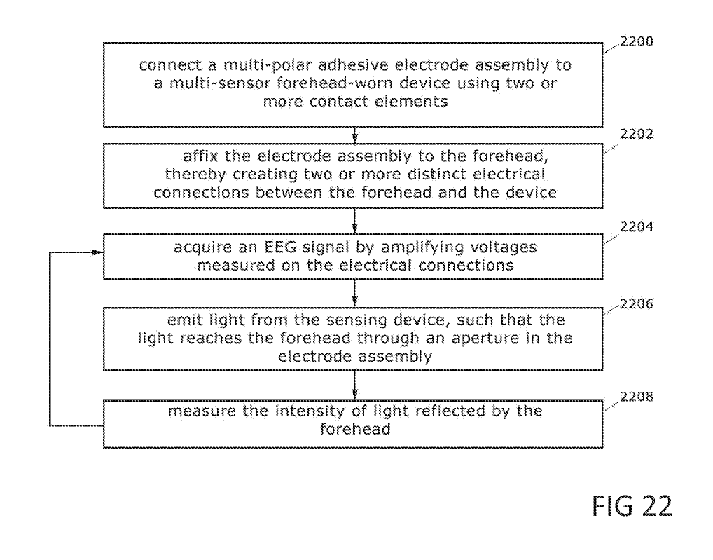

FIG. 22 shows the steps of a method for simultaneously acquiring EEG and pulse oximetry from the forehead of a person. In the electrode connection stage 2200, the adhesive electrode assembly 100 is connected to the EEG monitoring device 600. In the wearing stage 2202, the adhesive electrode assembly 100 is affixed to the forehead of a wearer. In the EEG acquisition stage 2204 the EEG signal is measured. In the light emission stage 2206, a light is emitted by the device 600, through a hole in the adhesive electrode assembly 100, towards the wearer's forehead. In the light measurement stage 2208, the light reflected by the wearer's forehead is measured, thereby acquiring an oximetry measurement. The stages are presented sequentially in this figure, although it is possible for the EEG signal and the oximetry signal to be measured at the same time, for instance if a single analog to digital converter is used by the CPU to simultaneously acquire measurements from two independently running sensors.

Other modifications and implementations will occur to those skilled in the art without departing from the spirit and the scope of the invention as claimed. Accordingly, the above description is not intended to limit the invention except as indicated in the following claims.

What is claimed is:

1. An adhesive-mountable head-wearable EEG apparatus for EEG monitoring, the apparatus comprising:
an EEG sensor capable of acquiring an EEG signal of a wearer of the wearable EEG apparatus;
a central processing unit capable of receiving the EEG signal from the EEG sensor;
a circuit board including the EEG sensor and the central processing unit;
an enclosing shell capable of enclosing the circuit board, the EEG sensor, and the central processing unit; and
an adhesive electrode assembly, the assembly including:
two or more gel electrodes capable of acquiring an EEG signal and capable of adhering to the forehead; and
two or more respective electrically conductive connection elements, each connection element being electrically connected to one of the gel electrodes, each connection element additionally being capable of being mated with a corresponding connection element on one of: the enclosing shell or the circuit board, so as to both electrically connect the circuit board to the electrodes, and structurally support the enclosing shell.

2. The apparatus of claim 1, wherein the adhesive electrode assembly further comprises at least one adhesive non-conductive area.

3. The apparatus of claim 1, wherein the enclosing shell has a left chamfered edge and a right chamfered edge, so as to reduce lateral forces on the enclosing shell when the apparatus is worn during sleep.

4. The apparatus of claim 1, wherein a horizontal width of the enclosing shell is no greater than a width that would bring the edge of the enclosing shell in contact with a sleep surface supporting the wearer before the nose of the wearer contacts the sleep surface.

5. The apparatus of claim 1, wherein the depth of the enclosing shell is no more than 2 cm.

6. The electrode assembly of claim 1, wherein the connection elements are snap fasteners.

7. The electrode assembly of claim 1, wherein the connection elements are magnets.

8. The electrode assembly of claim 1, the adhesive electrode assembly further comprising:
an aperture capable of allowing light to be directed towards and reflected by the forehead of the wearer, so as to allow a reflectance oximetry sensor to acquire oximetry measurements from the forehead while the electrode assembly is affixed to the forehead.

9. The electrode assembly of claim 1, wherein the gel electrodes are Ag—AgCl gel electrodes.

10. A method of simultaneously affixing and electrically connecting a forehead-worn EEG device to a person's forehead, comprising:
connecting an adhesive electrode assembly to the forehead-worn EEG device using two or more contact elements; and
affixing the adhesive electrode assembly to the forehead, thereby creating two or more respective electrical connections between the person's forehead and the forehead-worn EEG device.

11. The method of claim 10, wherein the contact elements are snap fasteners.

12. The method of claim 10, wherein the contact elements are magnets.

13. The method of claim 10, additionally comprising:
capturing a headband between the forehead-worn EEG device and the electrodes, so as to enable the headband to support the electrode assembly and ensure its adherence to the forehead.

14. A method of simultaneously acquiring EEG and pulse oximetry from the forehead of a person, the method comprising:
connecting a multi-polar adhesive electrode assembly to a multi-sensor forehead-worn device using two or more contact elements;
affixing the electrode assembly to the forehead, thereby creating two or more distinct electrical connections between the forehead and the device;
acquiring an EEG signal by amplifying voltages measured on the electrical connections;
emitting light from the sensing device, such that the light reaches the forehead through an aperture in the electrode assembly; and
measuring the intensity of light reflected by the forehead.
